(19) United States
(12) Patent Application Publication
(10) Pub. No.: US 2014/0272232 A1
(43) Pub. Date: Sep. 18, 2014

(54) ANTITHROMBIC COATINGS AND USES THEREOF

(71) Applicant: Bard Access Systems, Inc., Salt Lake City, UT (US)

(72) Inventor: Giridhar Thiagarajan, Salt Lake City, UT (US)

(21) Appl. No.: 14/211,212

(22) Filed: Mar. 14, 2014

Related U.S. Application Data
(60) Provisional application No. 61/799,537, filed on Mar. 15, 2013.

(51) Int. Cl. A61L 33/08; A61L 33/04; C09D 105/00

(52) U.S. Cl. CPC A61L 33/08; C09D 105/00; A61L 33/04
USPC 428/36.91; 428/474.7; 428/476.3; 428/476.9; 428/474.4; 428/500; 428/522; 428/523; 428/532; 427/2.24

(57) ABSTRACT

Provided are multi-layer antithrombic coatings comprising an outermost layer of an antithrombic agent, such as a heparin conjugate, bound to a penultimate polymeric layer comprising cationic polymer species that is bound alternating polymeric layers comprising anionic polymer species and cationic polymer species, and uses thereof.

### RELATED APPLICATIONS

This application claims the benefit of U.S. Provisional Application No. 61/799,537, filed Mar. 15, 2013, the contents of which are hereby incorporated by reference in their entirety.

### TECHNICAL FIELD

The present disclosure relates to the use of layered synthetic polymers for the immobilization of antithrombic materials to substrates. The present disclosure also relates to methods of preparing biocompatible surfaces on medical devices, and medical devices comprising such coatings.

### DESCRIPTION OF THE FIGURES

FIG. 1 is a representation of a six-layer antithrombic coating of the prior art, with layers 1-6 applied to a substrate.

FIG. 2 is a representation of a six-layer antithrombic coating according to certain embodiments of the disclosure, with layers 1-6 applied to a substrate.

FIG. 3 is a representation of a six-layer antithrombic coating according to certain embodiments of the disclosure, with layers 1-6 applied to a substrate, and depicting how partially-substituted underlying layers become more fully-substituted in the outermost layers.

### DETAILED DESCRIPTION

I. Definitions

As used herein, “substrate,” refers to a surface to which the disclosed antithrombic coatings are applied.

As used herein, “first,” when used in the context of a “first polymeric layer,” refers to the polymeric layer comprising a cationic polymer species that is closest to, and in contact with, a substrate.

As used herein, “second,” when used in the context of a “second polymeric layer,” refers to the polymeric layer comprising an anionic polymer species that is in contact with, and lies between, a first polymeric layer and third polymeric layer.

As used herein, “third” and “fourth,” when used in the context of a “third polymeric layer” and a “fourth polymeric layer,” refer to additional polymeric layers comprising cationic and anionic polymer species, respectively, and following the alternating ordering as laid out above for the first and second polymeric layers.

As used herein, “penultimate,” when used in the context of a “penultimate polymeric layer,” refers to the polymeric layer comprising a cationic polymer species that is located between an underlying polymeric layer comprising an anionic polymer species and the outermost layer of the disclosed antithrombic coatings. As such the penultimate polymeric layer is closest to, and in contact with, the outermost layer, which comprises an anionic antithrombic agent. In the 2-layer antithrombic coatings disclosed, the penultimate polymeric layer is in contact with, and lies between, the substrate and the outermost layer, which comprises an anionic antithrombic agent. In the 4-layer antithrombic coatings disclosed, the penultimate polymeric layer is in contact with, and lies between, a second polymeric layer and the outermost layer, which comprises an anionic antithrombic agent. In such embodiments, the penultimate polymeric layer can also be referred to as the third polymeric layer. In the 6-layer antithrombic coatings disclosed, the penultimate polymeric layer is in contact with, and lies between, a fourth polymeric layer and the outermost layer, which comprises an anionic antithrombic agent. In such embodiments, the penultimate polymeric layer can also be referred to as the fifth polymeric layer. In some embodiments, the penultimate polymeric layer comprises synthetic cationic polymer species (e.g., polylysine, polyornithine, chitosan, polyimines (e.g., poly ethylenimine), poly(amido amine)-amine terminated, polyallylamine, polyarginine, polyhistidine, and polyvinylpyrrolidone) capable of interacting electrostatically with the outer layer and the underlying (either second or fourth) layer.

As used herein, “outer,” when used in the context of an “outer layer,” refers to the outermost layer of the disclosed antithrombic coatings, and may comprise an anionic antithrombic agent. In the 2-layer antithrombic coatings disclosed, the outer layer can also be referred to as the second polymeric layer. In the 4-layer antithrombic coatings disclosed, the outer layer can also be referred to as the fourth polymeric layer. In the 6-layer antithrombic coatings disclosed, the outer layer can also be referred to as the sixth polymeric layer. In all embodiments, the outer layer is the layer most distant from the substrate and in contact with the immediate environment in which substrates having the disclosed antithrombic coatings are used or located, or through which biological fluids flow when the substrate is that of a lumenal device, such as a catheter.

As used herein, “polymer species” refers to a particular class of polymeric molecules. As such, the term “polymer species” is not meant to imply that all molecules within the “polymer species” are identical, but rather that they all fall within a particular class of polymeric molecules. However, in some embodiments, all molecules within a “polymer species” can be identical.

As used herein, “interacting electrostatically,” or “electrostatically interacts,” refers to the ionic bonding between the oppositely-charged groups on oppositely-charged polymer species. In the disclosed antithrombic coatings, discrete alternating polymeric layers comprising either cationic polymer species or anionic polymer species, interact electrostatically with each other. In the disclosed antithrombic coatings, the outermost layer comprising an anionic antithrombic agent electrostatically interacts with the penultimate polymeric layer, which comprises a cationic polymer species.

As used herein, “anionic antithrombic agent” or “antithrombic agent” refers to a biochemical entity that resists inducing thrombosis. In some embodiments, the “anionic antithrombic agent” or “antithrombic agent” refers to a biochemical entity that resists adhesion by platelets. In some embodiments the anionic antithrombic agent of the disclosed antithrombic coatings is heparin, or some derivative thereof. In some embodiments the anionic antithrombic agent of the disclosed antithrombic coatings is a heparin conjugate. In some embodiments the anionic antithrombic agent is a heparin conjugate such as the heparin conjugates disclosed in U.S. Pat. No. 5,529,986. Specifically, in those embodiments where the anionic antithrombic agent is a heparin conjugate such as the heparin conjugates disclosed in U.S. Pat. No. 5,529,986, the anionic antithrombic agent is a water-soluble conjugate having antithrombin-binding activity comprising a biologically inert carrier in the form of a substantially straight-chained organic polymer selected from the group consisting of polylysine, polyornithine, a polysaccharide and an aliphatic polymer, having chemically reactive groups distributed along the polymer backbone chain, and at least 30 molecules of sulphated glycosaminoglycan anchored to the chemically reactive groups through covalent bonds, wherein each sulphated glycosaminoglycan molecule is bound to the polymer backbone chain via a single point of attachment in a part of the sulphated glycosaminoglycan molecule that is not responsible for said antithrombin-binding activity, such that after anchoring of said molecule of sulphated glycosaminoglycan to said chemically reactive group, the molecule of sulphated glycosaminoglycan retains said antithrombin-binding activity.

As used herein, “biocompatible surface” or “biocompatible surfaces” refers to substrates having antithrombic coatings that are well tolerated by living mammalian organisms, living mammalian tissues or living mammalian cells when such biocompatible surfaces are contacted by such living organisms, tissues or cells. As such, biocompatible surfaces resist the induction of thrombosis, and resist inducing immune responses.

As used in this specification and the appended claims, the singular forms “a,” “an,” and, “the” include plural referents unless the context clearly dictates otherwise. Thus, for example, reference to “a device” may include one or more of such devices, reference to “a polymer species” may include reference to one or more types polymer species, and reference to “a synthetic aliphatic polyamine” may include reference to one or more of such compounds.

As used herein, “medical device” refers to any article of manufacture intended for use in medical or surgical procedures, including, but not limited to devices that are to be implanted within human or mammalian patients, such as interluminal implantation devices, or devices that come in direct contact with bodily fluids, including blood.

As used herein, “medical catheter” or “catheter” refers to a medical device that includes a flexible shaft, which contains one or more lumens which may be inserted into a subject for introduction of material (e.g., fluids, nutrients, medications, blood products, etc.), monitoring of the subject (e.g., pressure, temperature, fluid); and removal of material (e.g., body fluids), or any combination thereof. A catheter may further include various accessory components such as extension tubes, fittings, over molded junction hub, and so forth. A catheter may also have various tip and shaft features including holes, splits, tapers, overmolded tips or bumps, and so forth.

As used herein, “vascular access device” refers to a device that provides access to the circulatory system, typically the central circulatory system. Hence vascular access devices include both venous access devices or arterial access devices, such as indwelling catheters, cannulas, or other instruments used to obtain venous or arterial access. Such devices can be used to administer fluids and medications, monitor pressure, and collect blood or plasma samples.

As used herein, “venous access device” refers to a device that provides access to the venous circulation, typically the central venous circulation system. This includes but is not limited to central venous catheters, peripherally inserted venous catheters, ports, and dialysis catheters. Venous access devices may remain in place from days to years. The typical construction of a venous access catheter includes a flexible shaft with one or multiple lumens with various tips, splits, tapers, and so forth, that is connected by a junction hub to extension tubes with luer fitting for attachment to other devices.

As used herein, “central venous catheter” refers to a catheter with its tip placed directly in the central venous circulation system. These include any device, whether wholly implanted or partially implanted that delivers medication to the central parts of the heart, such as the central vena cava.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 micron to about 5 microns” should be interpreted to include not only the explicitly recited values of about 1 micron to about 5 microns, but also include individual values and sub-ranges within the indicated range. Thus, included in this numerical range are individual values such as 2, 3.5, and 4 and sub-ranges such as from 1-3, from 2-4, and from 3-5, etc.

This same principle applies to ranges reciting only one numerical value. For example, a range of values designated as less than 5, includes ranges less than 4 and less than 3. Furthermore, such an interpretation should apply regardless of the breadth of the range or the characteristics being described.

The embodiments described below are not intended to be exhaustive or to limit the invention to the precise forms disclosed in the following detailed description. Rather, the embodiments are chosen and described so that others skilled in the art can appreciate and understand the principles and practices of the present invention.

All publications and patents mentioned herein are hereby incorporated by reference. The publications and patents disclosed herein are provided solely for their disclosure. Nothing herein is to be construed as an admission that the inventors are not entitled to antedate any publication and/or patent, including any publication and/or patent cited herein.

### II. Antithrombic Coatings

The present disclosure provides novel antithrombic coatings and uses thereof, as well as methods for applying such coatings to substrates to create biocompatible surfaces. The antithrombic coatings disclosed comprise synthetic polymeric layers of alternating net charge for the immobilization of antithrombic materials to substrates. The individual layers that comprise the disclosed coatings are discrete layers comprising an assembly of polymer species having either a net positive charge or a net negative charge, and interacting electrostatically with the polymer species in adjacent polymeric layers. The polymeric layers comprised of polymer species having a net positive charge are referred to as “cationic polymeric layers,” or “cationic layers.” Whereas, the layers comprised of polymer species having a net negative charge are referred to as “anionic polymeric layers,” or “anionic layers.”

Cationic polymeric layers can comprise any suitable polymer species having a net positive charge. In some embodiments the cationic polymeric layers comprise cationic polymer species that are water soluble. In some embodiments the cationic polymeric layers comprise cationic polymer species are as described in U.S. Pat. No. 5,529,986. In particular embodiments the cationic polymer species are chosen from polylysine, polyornithine, chitosan, polyimines (e.g., poly ethylenimine), poly (amido amine)-amine terminated, polyallylamine, polyarginine, polyhistidine, and polyvinylpyrrolidone. In some embodiments the average molecular weight of the cationic polymer species ranges from about 40,000 to about 80,000 daltons. In particular embodiments the average molecular weight of the cationic polymeric species ranges from about 20,000 to about 100,000 daltons; from about 30,000 to about 90,000 daltons; from about 40,000 to about 80,000 daltons; and from about 50,000 to about 70,000 daltons.

In the antithrombic coatings disclosed, the first polymeric layer and the penultimate polymeric layer are cationic layers. In the 2-layered antithrombic coatings disclosed, the first polymeric layer is a cationic layer. In the 4-layered antithrombic coatings disclosed, the first polymeric layer and the third or penultimate polymeric layer are cationic layers. In the 6-layered antithrombic coatings disclosed, the first polymeric layer, the third polymeric layer, and the fifth or penultimate polymeric layer are cationic layers.

In the 4-layered antithrombic coatings disclosed, the first polymeric layer and the penultimate polymeric layer can each comprise the same cationic polymer species, or different cationic polymer species. In the 6-layered antithrombic coatings disclosed, the first polymeric layer, the third polymeric layer, and the penultimate polymeric layer can each comprise the same cationic polymer species, or different cationic polymer species. In the 6-layered antithrombic coatings disclosed, the first polymeric layer, the third polymeric layer, and the penultimate polymeric layer can each comprise different cationic polymer species. In some embodiments of the 6-layered antithrombic coatings disclosed, the first polymeric layer and the third polymeric layer can each comprise the same cationic polymer species, and the penultimate polymeric layer can comprise a cationic polymer species different from those comprising the first polymeric layer and third polymeric layer. In other embodiments of the 6-layered antithrombic coatings disclosed, the third polymeric layer and the penultimate polymeric layer can each comprise the same cationic polymer species, and the first polymeric layer can comprise cationic polymer species different from those comprising the third polymeric layer and penultimate polymeric layers. In still other embodiments of the 6-layered antithrombic coatings disclosed, the first polymeric layer and the penultimate polymeric layer can each comprise the same cationic polymer species, and the third polymeric layer can comprise cationic polymer species different from those comprising the first polymeric layer and penultimate polymeric layers.

Anionic polymeric layers can comprise any suitable polymer species having a net negative charge. In some embodiments the anionic polymeric layers comprise anionic polymer species that are water soluble. In some embodiments the anionic polymeric layers comprise anionic polymer species chosen from carboxyl terminated poly(amido amine) dendrimers, poly (acrylic acid)s, poly (acrylate)s, branched methacrylates, poly sulphonates, polystyrene sulfonates, poly phosphates, or carboxyl terminated dendrons. In particular embodiments the polymer species having a net negative charge are carboxyl terminated poly(amido amine) dendrimers. In some embodiments the average molecular weight of the anionic polymer species ranges from about 40,000 to about 80,000 daltons. In particular embodiments the average molecular weight of the anionic polymeric species ranges from about 20,000 to about 100,000 daltons; from about 30,000 to about 90,000 daltons; from about 40,000 to about 80,000 daltons; and from about 50,000 to about 70,000 daltons.

In the 2-layer antithrombic coatings disclosed, the second polymeric layer is the antithromic coating. However, in the 4-layered antithrombic coatings disclosed, the second polymeric layer is an anionic layer. And, in the 6-layered antithrombic coatings disclosed, the second polymeric layer and the fourth polymeric layer are anionic layers.

In the 6-layered antithrombic coatings disclosed, the second polymeric layer and the fourth polymeric layer can each comprise the same anionic polymer species, or can comprise different anionic polymer species having a net positive charge.

While not wishing to be bound by theory, the use of 4 or 6 polymeric layers in the disclosed antithrombic coatings results in a more uniform coating of antithrombic agents to substrates than might be achieved with only two polymeric layers (i.e., a penultimate polymeric layer and outer polymeric layer comprising an anionic antithrombic agent interacting electrostatically with said penultimate polymeric layer). While not wishing to be bound by theory, the use of 4 or 6 polymeric layers in the disclosed antithrombic coatings also results in a coating of antithrombic agents more resistant to erosion, degradation, or deterioration when exposed to biological fluids than antithrombic coatings comprising only a penultimate polymeric layer and outer polymeric layer. Consequently, the disclosed 4-layer and 6-layer antithrombic coatings provide an effective means to uniformly and resiliently immobilize antithrombic agents to substrates.

While not wishing to be bound by theory, the use in the polymeric layers of polymer species having a plurality of positively or negatively charged groups on each polymer molecule provides a plurality of sites for electrostatic interaction with an oppositely charged group on the polymer species comprising an adjacent polymeric layer. Practically, this means that after the first polymeric layer comprising cationic polymer species is bound to the substrate, the first polymeric layer provides a multiplicity of positively-charged binding groups to which the anionic polymer species of the second polymeric layer can bind. Similarly, the second polymeric layer comprising anionic polymer species electrostatically interacting with the first polymeric layer provides a greater multiplicity of negatively-charged binding groups to which the cationic polymer species of the third polymeric layer can bind, etc. Hence, with each additional polymeric layer being added to the previous polymeric layer, the numbers of charged groups available for electrostatic interaction increases. Consequently, with each successive layer the polymeric species making up the subsequent layer can become more tightly packed, so that the antithrombic outermost coating is contiguous, even if underlying layers are not.

Since the penultimate polymeric layer comprises a cationic polymer species, it provides a plurality of positively-charged binding groups to which the anionic antithrombic agent of the outer polymeric layer can bind.

In some embodiments the anionic antithrombic agent of the outer polymeric layer is heparin, or a heparin conjugate. In certain embodiments the anionic antithrombic agent of the outer polymeric layer is a macromolecular heparin conjugate. In certain embodiments the anionic antithrombic agent of the outer polymeric layer is a macromolecular heparin conjugate as disclosed in U.S. Pat. No. 5,529,986. As noted above, in those embodiments where the anionic antithrombic agent is a heparin conjugate such as the heparin conjugates disclosed in U.S. Pat. No. 5,529,986, the anionic antithrombic agent is a water-soluble conjugate having antithrombin-binding activity comprising a biologically inert carrier in the form of a substantially straight-chained organic polymer selected from the group consisting of polylysine, polyornithine, a polysaccharide and an aliphatic polymer, having chemically reactive groups distributed along the polymer backbone chain, and at least 30 molecules of sulphated glycosaminoglycan anchored to the chemically reactive groups through covalent bonds, wherein each sulphated glycosaminoglycan molecule is bound to the polymer backbone chain via a single point of attachment in a part of the sulphated glycosaminoglycan molecule that is not responsible for said antithrombin-binding activity, such that after anchoring of said molecule of sulphated glycosaminoglycan to said chemically reactive group, the molecule of sulphated glycosaminoglycan retains said antithrombin-binding activity.

In other embodiments, the anionic antithrombic agent of the outer polymeric layer is any glycosaminoglycan besides heparin, such as, for instance heparin sulphate, dermatan sulphate or chondroitin sulphate, as a covalently bound polymer, or a covalently bound conjugate.

In some embodiments the outer polymeric layer of the disclosed antithrombic coatings is the only layer in contact with the environment in which substrates having the disclosed antithrombic coatings are used or located. In some embodiments, the “outer” polymeric layer is the layer in contact with body fluid, whether it is the exterior of a device in contact with biological fluid, and/or the luminal surface which may be exposed to biological fluid. For example, the disclosed antithrombic coatings may be applied to the lumens of devices like catheters such that the outer polymeric layer of the disclosed antithrombic coatings is the luminal surface, and is the only layer in contact with biological fluids that flow through the catheter.

### III. Applications for Antithrombic Coatings

The present disclosure provides reagents and methods for providing an antithrombic coating to the surface of an article, the coating including layered polymeric materials and an antithrombic agent coupled to the layered polymeric material. The coating can be formed on a variety of articles, wherein it is desired to have the functional properties of an antithrombic agent on a surface of the article. The coatings of the present disclosure can be formed on articles used in various technologies, including, but not limited to, articles that are used in medical technologies including implantable medical devices, surgical equipment, and surgical instruments; assay instrumentation and products, such as biosensor-based systems, chemiluminescence detection systems, immunoassay systems; assay plates, including 1536, 384, and 96 well plates; solid supports; microbiology equipment such as fermentation equipment and bacteriological testing equipment; tubing; cell biology articles, such as cell assay kits; cell biology equipment, such as tissue processing articles, flow cytometry articles, and screening articles; cell culture articles such as culture jars, cell collection systems, cell harvesters, cell separation articles, culture dishes, culture flasks, culture plates, culture roller bottles, culture slides, and culture tubes; bioreactors; fermenters; hollow fiber systems; perfusion systems; suspension systems; chromatography and separation systems, such as affinity columns and biomolecular columns; detectors, such as amperometric detectors, chemiluminescence detectors, electrochemical detectors, fluorescence detectors, and MALDI-TOF mass spec; drug discovery systems, such as articles used in high throughput systems; filters and filtration equipment, including bacteriological filters, glass fibers, affinity membranes, microbial membranes, microfilters, tissue culture; genomic and proteomic system articles, such as microarray articles including slides, chips, and microfluidic articles; immunochemical systems including ELISA and immunoassay kits; microscope slides and accessories; nucleic acid equipment including automated sequencers; nucleic acid analysis kits; protein analysis equipment; ampules; glassware; petri dishes; test tubes; vials; and plastic and micro pipets.

In particular, and as disclosed in more detail below, the coatings of the present disclosure can be formed on medical devices, including, but not limited to, catheters, stents, needless connectors, vascular grafts, catheter balloons, sutures, staples, anastomosis devices, vertebral disks, bone pins, suture anchors, haemostatic barriers, clamps, screws, plates, clips, vascular implants, tissue scaffolds, bone substitutes, intraluminal devices, and vascular supports.

In some embodiments, the coatings of the present disclosure can be formed on medical devices wherein the device is implantable into a mammalian lumen.

The coatings disclosed can also be formed on a wide variety of materials used to fabricate an article or device. The materials to form the structure of the article are referred to herein as “article materials” or “device materials” whereas the materials used to form the polymeric coatings herein referred to as “coating materials.” In many cases, the article can be formed from one or more biomaterial(s) if the coated article is to be placed in contact with a biological fluid or tissue (such as being implanted in the body).

Examples of materials which can be used to form the article onto which the coating can be added include synthetic polymers, including oligomers, homopolymers, and copolymers resulting from either addition or condensation polymerizations. Examples of suitable addition polymers include, but are not limited to, acrylics such as those polymerized from methyl acrylate, methyl methacrylate, hydroxyethyl methacrylate, hydroxyethyl acrylate, acrylic acid, methacrylic acid, glyceryl acrylate, glyceryl methacrylate, methacrylamide, and acrylamide; vinyls such as ethylene, propylene, vinyl chloride, vinyl acetate, vinyl pyrrolidone, and vinylidene difluoride.

The materials can be used to fabricate a variety of implantable medical devices. The medical device can be any device that is introduced temporarily or permanently into a mammal for the prophylaxis or treatment of a medical condition, or for diagnosis or treatment of the mammal into which the medical device is implanted. These devices include any that are introduced subcutaneously, percutaneously or surgically to rest within an organ, tissue, or lumen of an organ, such as arteries, veins, ventricles, or atria of the heart.

The disclosed antithrombic coatings can be applied to the surface of a variety of implantable medical devices. In some aspects the coatings that are formed provide a biocompatible surface to the implantable medical device. The biocompatible surface can enhance the ability of the medical device to function or exist in contact with biological fluid and/or tissue of a living organism with a net beneficial effect on the living organism, or at least a minimized negative effect on the living organism.

The disclosed antithrombic coatings can be formed on devices such as drug-delivering vascular stents; other vascular devices (e.g., grafts, catheters, valves, artificial hearts, heart assist devices, ventricular assist devices); implantable defibrillators; blood oxygenator devices; surgical devices; tissue-related materials; membranes; shunts for hydrocephalus; wound management devices; endoscopic devices; infection control devices; orthopedic devices; dental devices, urological devices; colostomy bag attachment devices; ophthalmic devices; glaucoma drain shunts; synthetic prostheses; intraocular lenses; respiratory, peripheral cardiovascular, spinal, neurological, dental, and ear/nose/throat devices (e.g., ear drainage tubes); renal devices; and dialysis (e.g., tubing, membranes, grafts).

The disclosed antithrombic coatings can be formed on other devices such self-expanding stents (e.g., made from nitinol), balloon-expanded stents (e.g., prepared from stainless steel), degradable coronary stents, non-degradable coronary stents, peripheral coronary stents, endovascular stents, intraaortic balloons, urinary catheters (e.g., surface-coated with antimicrobial agents), penile implants, sphincter devices, urethral devices, bladder devices, renal devices, vascular implants and grafts, intravenous catheters (e.g., treated with antithrombotic agents), needless connectors, vascular grafts, small diameter grafts, artificial lung catheters, electrophysiology catheters, pacemaker leads, anastomosis devices, vertebral disks, bone pins, suture anchors, haemostatic barriers, clamps, surgical staples/sutures/screws/plates/clips, atrial septal defect closures, electro-stimulation leads for cardiac rhythm management (e.g., pacer leads), glucose sensors (long-term and short-term), blood pressure and stent graft catheters, blood oxygenator tubing, blood oxygenator membranes, blood bags, birth control devices, breast implants; benign prostatic hyperplasia and prostate cancer implants, bone repair/augmentation devices, cartilage repair devices, orthopedic joint implants, orthopedic fracture repairs, tissue adhesives, tissue sealants, tissue scaffolds, CSF shunts, dental implants, dental fracture repair devices, implanted drug infusion tubes, intravitreal drug delivery devices, nerve regeneration conduits, oncological implants, electrostimulation leads, pain management implants, spinal/orthopedic repair devices, surgical blood salvage disposal sets, wound dressings, embolic protection filters, abdominal aortic aneurysm grafts, heart valves (e.g., mechanical, polymeric, tissue, percutaneous, carbon, sewing cuff), valve annuloplasty devices, mitral valve repair devices, vascular intervention devices, left ventricle assist devices, neuro aneurysm treatment coils, neurological catheters, left atrial appendage filters, central venous access catheters, hemodialysis devices, hemodialysis catheters, catheter cuff, anastomotic closures, vascular access catheters, cardiac sensors, intravascular sensors, uterine bleeding patches, urological catheters/stents/implants, in vitro diagnostics, aneurysm exclusion devices, neuropatches, Vena cava filters, urinary dilators, endoscopic surgical tissue extractors, atherectomy catheters, clot extraction catheters, PTA catheters, PTCA catheters, stylets (vascular and non-vascular), coronary guidewires, drug infusion catheters, esophageal stents, circulatory support systems, angiographic catheters, transition sheaths and dilators, coronary and peripheral guidewires, hemodialysis catheters, neurovascular balloon catheters, tympanostomy vent tubes, cerebrospinal fluid shunts, defibrillator leads, percutaneous closure devices, drainage tubes, thoracic cavity suction drainage catheters, electrophysiology catheters, stroke therapy catheters, abscess drainage catheters, biliary drainage products, dialysis catheters, central venous access catheters, and parental feeding catheters.

### IV. Methods of Applying Antithrombic Coatings

As noted above, each polymeric layer of the disclosed antithrombic coatings comprises an assembly of polymeric molecules (i.e., species), which are either cationic polymer species or anionic polymer species. Since the polymeric layers alternate in charge, with the first, third, and optionally fifth, polymeric layers having a net positive charge, and the second, and optionally fourth, polymeric layers having a net negative charge, each underlying polymeric layer serves to provide a surface on which the next polymeric layer can electrostatically interact and self-assemble. Consequently, the process by which the disclosed antithrombic coatings can be applied to a substrate can involve a series of steps in which each successive polymeric layer is self-assembled on the previously assembled polymeric layer.

One method for applying the disclosed antithrombic coatings is provided as Example 2, below.

### EXAMPLES

### Example 1

A six-layer antithrombic coating known from the prior art is depicted in FIG. 1. In this antithrombic coating, a macromolecular heparin conjugate is the anionic polymer species comprising the second, fourth and outermost polymeric layers. The first, third and fifth polymeric layers comprise a cationic polymer species, such as polylysine, polyornithine, chitosan, polyimines (e.g., poly ethylenimine), poly (amido amine)-amine terminated, polyallylamine, polyarginine, or polyhistidine.

An exemplary six-layer antithrombic coating as presently disclosed is depicted in FIG. 2. In contrast to the coating depicted in FIG. 1, this six-layer antithrombic coating employs a macromolecular heparin conjugate in only the outermost layer, where it serves as an antithrombic agent. Like the coating depicted in FIG. 1, the first, second and third polymeric layers in the coating of FIG. 2 comprise a cationic polymer species chosen from polylysine, polyornithine, chitosan, polyimines (e.g., poly ethylenimine), poly (amido amine)-amine terminated, polyallylamine, polyarginine, polyhistidine, and polyvinylpyrrolidone. Unlike the coating depicted in FIG. 1, the second and fourth polymeric layers in the coating of FIG. 2 comprise an anionic polymer species that is not a macromolecular heparin conjugate, but is another anionic polymer species that can be chosen from carboxyl terminated poly(amido amine) dendrimers, poly (acrylic acid)s, poly (acrylate)s, branched methacrylates, poly sulphonates, polystyrene sulfonates, poly phosphates, or carboxyl terminated dendrons.

While not wishing to be bound by theory, it is believed that polymer species that comprise each discrete layer self-assemble to form an ordered monolayer. Moreover, it is believed that the thermodynamic stability of the ordered, self-assembled, monolayer arises from the exclusion of waters of hydration from the substrate and/or underlying monolayers, from Van der Waals forces of interaction between the individual molecules of the polymer species used to form the monolayer, and from ionic interactions between the polymer species comprising one discrete monolayer with oppositely-charged polymer species of an adjacent monolayer. Additionally, while not wishing to be bound by theory, it is believed that the overall stability of the multilayer coatings of the present disclosure derives from a combination of the inherent stability of the self-assembled monolayers, and the intrinsic stability resulting from the ionic interactions between discrete monolayers. As discussed elsewhere, it is believed that the use of multiple monolayers of alternating net charge results in the outermost layer being more uniformly coated with the antithrombic agent, since underlying layers that may be partially-substituted with assemblies of polymer species become more fully-substituted as each subsequent layer self-assembles over a partially-substituted underlying layer, as depicted in FIG. 3. FIG. 3 depicts how the use of multiple layers of polymeric species with multiple charged groups, such as dendrimers, can result in increased degrees of substitution with each subsequent coating layer. The net result being that partially-substituted underlying layers become more fully-substituted in each subsequent layer, such that the outermost layer can be fully saturated, and uniformly coated with the antithrombic agent.

### Example 2

Provided below is a method for applying an antithrombic coating as disclosed herein to the exposed surfaces of a catheter. This same method can be used to apply an antithrombic coating as disclosed herein to the exposed surfaces of any suitable medical device. The method generally comprises the following steps:
a) Dip the catheter in a solution of ammonium persulfate;
b) Dip the catheter in a solution containing a cationic polymer species, such as polyethylene imine, to bind the first polymeric layer to the exposed surfaces of the catheter;
c) Dip the catheter in a solution containing an anionic polymer species, to provide a second polymeric layer containing an anionic polymer species electrostatically interacting with the cationic polymeric species of the first polymeric layer;
d) Dip the catheter in a solution containing a cationic polymer species, to provide a third polymeric layer containing an cationic polymer species electrostatically interacting with the anionic polymeric species of the second polymeric layer;
e) Dip the catheter in a solution containing a solution containing a macromolecular heparin conjugate, to provide an outer layer comprising an anionic antithrombic agent;
f) Rinse the catheter with borate buffer, pH 9, and water; and
g) Dry the catheter.

Optionally, after each of steps a) through e) above, the catheter can be drained and/or rinsed by dipping in deionized sterile water, saline, a buffer solution, or a buffered saline solution, and drained again before the next step is conducted. This draining and/or rinsing and draining step can improve the working life and/or effectiveness of each coating solution, so that the same reagent baths can be used to treat a greater number of catheters before the solution in the baths needs to be replaced.

The above method can be used to apply a 4-layer antithrombic coating as disclosed herein to the exposed surfaces of a catheter. To apply a 6-layer antithrombic coating, after step c), repeat steps b) and c) once, before proceeding to steps d)-g). Similarly, the above method can be used to apply a 2-layer antithrombic coating by simply omitting steps c) and d).

Optionally, after all of the coating steps have been completed, the catheter can be finally rinsed by dipping in deionized sterile water, saline, a buffer solution, or a buffered saline solution, and can then be drained and dried before packaging.

1. An antithrombic coating, comprising
a) a substrate;
b) a first polymeric layer comprising synthetic cationic polymer species bound to said substrate;
c) a second polymeric layer comprising synthetic anionic polymer species interacting electrostatically with said first polymeric layer;
d) a penultimate polymeric layer comprising synthetic cationic polymer species interacting electrostatically with said second polymeric layer; and
e) an outer layer comprising an anionic antithrombic agent interacting electrostatically with said penultimate polymeric layer.

2. The antithrombic coating of claim 1, further comprising a third polymeric layer and a fourth polymeric layer situated between said second polymeric layer and said penultimate polymeric layer, wherein said third polymeric layer comprises synthetic cationic polymer species interacting electrostatically with said second polymeric layer, and said fourth polymeric layer comprises synthetic anionic polymer species interacting electrostatically with said third polymeric layer, and said penultimate polymeric layer electrostatically interacts with said fourth polymeric layer.

3. The antithrombic coating of claim 2, wherein one or more of said first polymeric layer, said third polymeric layer, and said penultimate polymeric layer comprises a synthetic cationic polymer species selected from polylysine, polyornithine, chitosan, polyimines (e.g., poly ethylenimine), poly (amido amine)-amine terminated, polyallylamine, polyarginine, polyhistidine, or polyvinylpyrrolidone.

4. The antithrombic coating of claim 3, wherein said penultimate polymeric layer comprises polylysine.

5. The antithrombic coating of claim 2, wherein one or more of said second polymeric layer and said fourth polymeric layer comprise synthetic anionic polymer species chosen from carboxyl terminated poly(amido amine) dendrimers, poly(acrylic acid)s, poly (acrylate)s, branched methacrylates, poly sulphonates, polystyrene sulfonates, poly phosphates, or carboxyl terminated dendrons.

6. The antithrombic coating of claim 2, wherein said first polymeric layer comprises the same cationic polymer species of said penultimate polymeric layer, and/or said third polymeric layer.

7. The antithrombic coating of claim 2, wherein said first polymeric layer comprises a different cationic polymer species than said penultimate polymeric layer, and/or said third polymeric layer.

8. The antithrombic coating of claim 2, wherein said penultimate polymeric layer comprises the same cationic polymer species of said first polymeric layer and/or said third polymeric layer.

9. The antithrombic coating of claim 2, wherein said penultimate polymeric layer comprises cationic polymer species that are different from the cationic polymer of said first and/or said third polymeric layer.

10. The antithrombic coating of claim 2, wherein said second polymeric layer and said fourth polymeric layer comprise the same anionic polymer species.

11. The antithrombic coating of claim 2, wherein said second polymeric layer and said fourth polymeric layer comprise different anionic polymer species.

12. The antithrombic coating of claim 2, wherein said outer layer comprises heparin, a heparin conjugate, or a macromolecular heparin conjugate.

13. The antithrombic coating of claims 12, wherein said outer layer comprises a macromolecular heparin conjugate that is a water-soluble conjugate having antithrombin-binding activity comprising a biologically inert carrier in the form of a substantially straight-chained organic polymer selected from the group consisting of polylysine, polyornithine, a polysaccharide and an aliphatic polymer, having chemically reactive groups distributed along the polymer backbone chain, and at least 30 molecules of sulphated glycosaminoglycan anchored to the chemically reactive groups through covalent bonds, wherein each sulphated glycosaminoglycan molecule is bound to the polymer backbone chain via a single point of attachment in a part of the sulphated glycosaminoglycan molecule that is not responsible for said antithrombin-binding activity, such that after anchoring of said molecule of sulphated glycosaminoglycan to said chemically reactive group, the molecule of sulphated glycosaminoglycan retains said antithrombin-binding activity.

14. A medical device having the antithrombic coating of claim 2.

15. The medical device of claim 14, wherein said medical device is chosen from a catheter, a stent, a needless connector, a vascular graft, a catheter balloon, a suture, a staple, an anastomosis device, a vertebral disk, a bone pin, a suture anchor, a haemostatic barrier, a clamp, a screw, a plate, a clip, a vascular implant, a tissue scaffold, a bone substitute, an intraluminal device, and a vascular support.

16. The device of claim 15, wherein the device is implantable into a mammalian lumen.

17. A method of applying a 4-layer antithrombic coating of claim 1 to a medical device comprising:
a) dipping the medical device in a solution of ammonium persulfate;
b) dipping the medical device in a solution containing a cationic polymer species to bind the first polymeric layer to the exposed surfaces of the medical device;
c) dipping the medical device having the first polymeric layer in a solution containing an anionic polymer species, to provide a second polymeric layer containing an anionic polymer species electrostatically interacting with the cationic polymeric species of the first polymeric layer;
d) dipping the medical device having the first and second polymeric layer in a solution containing a cationic polymer species, to provide a third and penultimate polymeric layer containing an cationic polymer species electrostatically interacting with the anionic polymeric species of the second polymeric layer; and
e) dipping the medical device having the first, second and penultimate polymeric layer in a solution containing a solution containing an anionic antithrombic agent, to provide an outer layer comprising an anionic antithrombic agent electrostatically interacting with the cationic polymer species of the third and penultimate polymeric layer.

18. The method of claim 17, wherein after each of steps a) through d), the medical device is drained and optionally rinsed before the next step is conducted.

19. A method of applying a 6-layer antithrombic coating of claim 2 to a medical device comprising the following steps:
a) dipping the medical device in a solution of ammonium persulfate;
b) dipping the medical device in a solution containing a cationic polymer species, cationic polymer species to bind the first polymeric layer to the exposed surfaces of the medical device;
c) dipping the medical device having the first polymeric layer in a solution containing an anionic polymer species, to provide a second polymeric layer containing an anionic polymer species electrostatically interacting with the cationic polymeric species of the first polymeric layer;
d) dipping the medical device having the first and second polymeric layer in a solution containing a cationic polymer species, to provide a third polymeric layer containing an cationic polymer species electrostatically interacting with the anionic polymeric species of the second polymeric layer;
e) dipping the medical device having the first, second and third polymeric layer in a solution containing an anionic polymer species, to provide a fourth polymeric layer containing an anionic polymer species electrostatically interacting with the cationic polymeric species of the third polymeric layer;
f) dipping the medical device having the first, second third, and fourth polymeric layer in a solution containing a cationic polymer species, to provide a fifth and penultimate polymeric layer containing an cationic polymer species electrostatically interacting with the anionic polymeric species of the fourth polymeric layer;
g) dipping the medical device having the first, second, third, fourth, and penultimate polymeric layer in a solution containing a solution containing an anionic antithrombic agent, to provide an outer layer comprising an anionic antithrombic agent electrostatically interacting with the cationic polymer species of the fifth and penultimate polymeric layer.

20. The method of claim 19, wherein after each of steps a) through f), the medical device is drained and optionally rinsed before the next step is conducted.
